(19) United States
(12) Patent Application Publication
(10) Pub. No.: US 2025/0332275 A1
(43) Pub. Date: Oct. 30, 2025

(54) Branched Polypeptide Carrier for Efficient Nucleic Acid Delivery and Its Variants

(71) Applicant: SUZHOU HEALIRNA BIOTECHNOLOGY CO., LTD., Suzhou (CN)

(72) Inventors: Qingqing LU, Suzhou (CN); Dan XIA, Suzhou (CN); Cheng YI, Suzhou (CN); Youzhen GE, Suzhou (CN); Hongpeng HU, Suzhou (CN); Jianhao JIANG, Suzhou (CN); Tianyi ZHONG, Suzhou (CN); Lixia ZHANG, Suzhou (CN); Kan JIA, Suzhou (CN); Yuyang ZHANG, Suzhou (CN)

(73) Assignee: SUZHOU HEALIRNA BIOTECHNOLOGY CO., LTD., Suzhou (CN)

(21) Appl. No.: 19/059,348

(22) Filed: Feb. 21, 2025

Related U.S. Application Data
(63) Continuation of application No. PCT/CN2023/111747, filed on Aug. 8, 2023.

(30) Foreign Application Priority Data
Aug. 23, 2022 (CN) 202211014569.4

(51) Int. Cl. A61K 47/64; C07K 7/06; C07K 7/08; C12N 15/87

(52) U.S. Cl. CPC A61K 47/641; C07K 7/06; C07K 7/08; C12N 15/87

(57) ABSTRACT

The disclosure provides a branched structure polypeptide peptide carrier and its variations. The branched structure peptide has the following sequence formula:

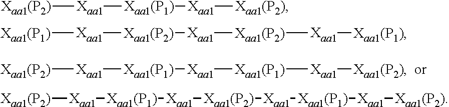

The technical solution of the disclosure enables efficient delivery of nucleic acids to tissues and organs in vivo for targeted therapy. The polypeptides have several times more conformational flexibility and affinity than macromolecular drugs (proteins and antibodies). The branched structure polypeptides possess long-lasting in vivo stability while maintaining strong affinity and minimal toxicity. By forming stable nanocomplexes or nanoparticles through electrostatic interactions with nucleic acid molecules, they can facilitate the delivery of nucleic acid drugs and their stable release inside cells, thereby enhancing the activity of nucleic acid-based therapeutics.

### CROSS-REFERENCE TO RELATED APPLICATIONS

This application is a continuation and claims priority to International Application No. PCT/CN2023/111747, filed on Aug. 8, 2023, which claims priority to Chinese Patent Application No. 202211014569.4, filed Aug. 23, 2022. The disclosure of the above-described applications is hereby incorporated by reference in their entirety.

### REFERENCE TO AN ELECTRONIC SEQUENCE LISTING

The contents of the electronic sequence listing (ZL0082-0002-US Sequence Listing.xml; Size: 50,418 bytes; and Date of Creation: Feb. 21, 2025) is hereby incorporated by reference in its entirety.

### FIELD OF THE INVENTION

The present invention pertains to the field of nucleic acid delivery, particularly to a branched polypeptide carrier for efficient nucleic acid delivery and its variants.

### BACKGROUND TECHNOLOGY

Nucleic acid-based therapeutics, including plasmid DNA (pDNA), small interfering RNA (siRNA), microRNA (miRNA), antisense oligonucleotides, messenger RNA (mRNA), and aptamers, can alter cellular genetic information, thereby inducing biological effects in cells and organisms. However, the transition of nucleic acids from extracellular to intracellular environments faces challenges such as degradation, precipitation, and protein binding. Polypeptide carriers can protect nucleic acids from degradation, precipitation, and protein interactions, ensuring their functionality within the body.

Nucleic acids typically exhibit a negative charge under physiological conditions. Cationic compounds can electrostatically bind with nucleic acids, compressing and protecting them from enzymatic degradation. These compounds facilitate fusion with cell membranes, enabling nucleic acids to escape from endosomes. Common small-molecule cationic lipids, such as DOTAP (1,2-dioleoyl-3-trimethylammonium-propane) and DDAB (dodecyl-dimethylammonium bromide), achieve moderate transfection efficiency in vitro but fail to meet expectations in vivo. Additionally, cationic polymers like poly-L-lysine, DEAE-dextran, polyethyleneimine (PEI), and chitosan possess a high density of positive charges. Following cellular internalization, their proton sponge effect causes water influx into endosomes, leading to endosomal rupture and the release of nucleic acids into the cytoplasm. Therefore, these polymers not only exhibit cytotoxicity, but also have low nucleic acid expression efficiency.

Research into disease-related genetic mechanisms has highlighted nucleic acid-based therapeutics as promising treatments for various diseases, gaining global attention. Several nucleic acid drugs have gained approval for clinical use. Nevertheless, rapid degradation, rapid clearance by liver and kidneys, low cellular uptake rates, and poor endosomal escape severely hinder their clinical application. Efficient delivery to target cells is critical for drug efficacy. Polypeptides offer numerous advantages for gene delivery. Most polypeptides drugs are derived from endogenous or natural peptides. Polypeptides serve important human physiological roles such as hormones, neurotransmitters, growth factors, and ion channel ligands. They have the advantages of safety, tolerance, effectiveness, and east of synthesis, etc. Compared to large molecules drugs (such as proteins and antibodies), polypeptides have more flexible conformations. For instance, cyclic peptides exhibit prolonged stability in vivo while maintaining high binding affinity and minimal toxicity.

Currently existing reliable methods in solid-phase synthesis can provide high-purity polypeptides with defined structures and can achieve secondary configurations with important biological activities, such as α-helices and β-sheets. Coupling with sequences or molecules with tissue-targeting properties produces targeted polypeptides. Through non-covalent interactions (including, e.g., hydrophobic interactions, electrostatic interactions, intermolecular hydrogen bonding, and TT-TT stacking, etc), polypeptides can self-assemble in certain order to form stable nanostructures. As bioidentical new generation biomaterials, polypeptides have advantageous biological and chemical characteristics. Among them, amphipathic polypeptide molecules have characteristics that resemble those of natural phospholipids, but with more diverse molecular structures, and can be assembled to form assembly structures with specific biological functions. Thus, through rational design of the amino acid sequence of the polypeptides, they can form stable nanocomplexes or nanoparticles with nucleic acid molecules through electrostatic interactions, enabling the delivery of nucleic acid-based drugs and their stable intracellular release, thereby enhancing drug activity.

As in vivo drug carriers or prodrugs, polypeptides expand their pharmaceutical applications through adsorption, encapsulation, or modification of target genes. For example, the specific binding of RGD peptides with integrin receptors can be used for targeted tumor therapy. Combining polypeptides with antibodies offers a new method in developing novel polypeptide therapeutic strategies. In antibody-peptide conjugate drugs, antibodies can act as targeting moieties, and peptides as effector moieties. The polypeptide conjugation method have strong therapeutic potential. Targeting mitochondria in tumor cells, KLAKLAKKLAKLAK, being an amphipathic α-helical cell-penetrating peptide and being pro-apoptotic, can be effective in anti-tumor applications. Melittin, a major component of bee venom, can target-deliver RNA interference (RNAi) to liver cells.

The primary challenge in peptide delivery is the degradation of peptides. Cyclization reduces proteolytic cleavage, and branched peptides are characterized by half-life improvement, offering promise in cancer targeting. Current strategies to enhance polypeptide drug stability involve conjugation with carriers (e.g., liposomes or solid nanoparticles) or constructing multi-branched polypeptides to form nanoscale formulations with specificity and stability. Compared to linear polypeptides, branched polypeptides have defined chemical structures and greater stability.

### SUMMARY OF THE INVENTION

Based on the above background, the present invention provides a branched structure polypeptide carrier with high gene delivery efficiency and stable structure for effectively delivering nucleic acids, and its variations. The technical solution adopted is as follows:

According to one aspect of the present invention, a branched structure polypeptide is provided with the following sequence formula:

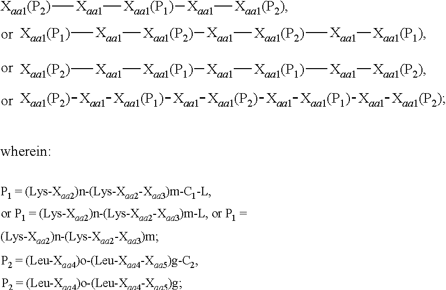

Xₐₐ₁ represents Aspartic acid (Asp), Glutamic acid (Glu), or Lysine (Lys);
Xₐₐ₂, Xₐₐ₃ each represents any of Aspartic acid (Asp), Glutamic acid (Glu), Lysine (Lys), Arginine (Arg), Alanine (Ala), Glycine (Gly), Serine (Ser), Asparagine (Asn), or Glutamine (Gln);
Xₐₐ₄, Xₐₐ₅ each represents any of Glycine (Gly), Serine (Ser), Tyrosine (Tyr), Threonine (Thr), Alanine (Ala), Valine (Val), Leucine (Leu), Isoleucine (Ile), Proline (Pro), Phenylalanine (Phe), Tryptophan (Trp), or Methionine (Met);
C₁ and C₂ are Cysteine (Cys);
L is an optional ligand, which may be present or absent, and can independently be selected from ligands such as spermine or spermidine, polylysine, polyarginine, MPG peptides, HIV-binding peptides Tat, SAP peptides, MAP peptides, KALA peptides, FGF peptides, HSV peptides, RGD peptides, GLP-1 peptides, PEP-1 peptides, signal peptides or signal sequences, cytokines, growth factors, hormones, small molecular drugs, carbohydrates, therapeutic active proteins or peptides, antigens or antigen epitopes, antibodies, antibody receptor ligands, synthetic ligands, receptor inhibitors or antagonists, immunostimulatory proteins or peptides, protamine, nucleolar proteins, transferrin, polycationic peptide sequences, polyamines, peptide nucleic acids, nucleic acid embedding agents, nuclear localization signals or sequences (NLS), cell-penetrating peptides, mannose, galactose, small molecular agonists, poly-L-lysine (PLL), basic peptides, calcitonin peptides, lactoferrin, histones, VP22-derived or similar peptides, or VP22 (herpes simplex);
m, n, o, g may independently be selected from any of the values 0, 1, 2, 3, 4, 5, 6, 7, 8, 9, and 10;

The branched structure polypeptide contains a length of 20 to 150 amino acid residues; The branched structure polypeptide contains at least one disulfide bridge;

The branched structure includes a 2-branched, 3-branched, 4-branched, or 5 branched configuration.

According to another aspect of the invention, a branched structure composition is provided. The branched structure polypeptide composition comprises one branched structure described in the aforementioned technical solution used independently or multiple branched structure polypeptides described in the aforementioned technical solution used in combination.

According to yet another aspect of the invention, a branched structure polypeptide-nucleic acid complex is provided. The branched structure polypeptide-nucleic acid complex is formed by further allowing mixing the branched structure polypeptide composition with nucleic acids for it to include or carry one or more nucleic acids to form the branched structure polypeptide-nucleic acid complex.

In a further embodiment, the nucleic acids mentioned herein include naturally-occurring DNA, RNA, or DNA or RNA extracted or synthesized through artificial design. The DNA or RNA extracted or synthesized through artificial design includes small interfering RNA (siRNA), messenger RNA (mRNA), microRNA (miRNA), small activating RNA (saRNA), antisense nucleic acids, or immunostimulatory nucleic acids, etc.

In a further embodiment, the branched structure polypeptide-nucleic acid complex can effectively carry nucleic acids across animal and human cell membranes and release the nucleic acids.

In a further embodiment, the branched structure polypeptide-nucleic acid complex can be used in combination with drugs or therapeutic agents. For example, the drugs or agents is selected from peptides, antibodies, enzymes, hormones, anti-tumor drugs, anti-lipid drugs, antibiotics, anti-inflammatory agents, cytotoxic agents, cell growth inhibitors, immunomodulators, or neuroactive agents.

In a further embodiment, the branched structure polypeptide-nucleic acid complex contains complexes formed by the polypeptide and a C12-C18 alkyl group or a C6-C18 alkyl containing an olefinic bond.

In a further embodiment, the aforementioned branched structure polypeptide composition or complex may contain one or more surfactants, liposomes, lipidoids, ethosomes, transporters, phospholipids, or any combination thereof.

Preferably, the liposomes include, but are not limited to, commercial liposomes such as N-[1-(2,3-dioleoyloxy) propyl)]-N,N,N-trimethylammonium chloride (DOTAP-Cl), N-[1-(2,3-dioleyloxy) propyl)]-N,N,N-trimethylammonium chloride (DOTMA), distearoylphosphatidylcholine (DSPC), dioleoylphosphatidylcholine (DOPC), dipalmitoylphosphatidylcholine (DPPC), dioleoylphosphatidylglycerol (DOPG), dioleoylphosphatidylethanolamine (DOPE), 3,β-[N, (N′,N′-dimethylaminoethane)-carbamoyl]cholesterol (DC-chol), dimyristoylphosphatidylcholine (DMPC), dimyristoylphosphatidylethanolamine (DMPE), dioleoyldimethylammonium chloride (DODAC), 1-palmitoyl-2-oleoyl-sn-glycero-3-phosphocholine (POPC), 1,2-dioleoyl-3-trimethylammonium-propane (DOTAP), 1,2-dioleyloxy-3-dimethylamino-propane (DODMA), 1,2-dimyristoyl-rac-glycero-3-methoxy polyethylene glycol (DMG-PEG2000), 2-(spermidine amido)ethyl)-N,N-dimethyl-trifluoroacetic acid ammonium (DOSPA), 1,2-dioleoyl-3-dimethylammonium-propane (DODAP), 2,3-dioleoyloxy-N-[2-(spermidinecarboxyamido)ethyl]-N,N-dimethyl-1-trifluoroacetic-aminopropane (DOSPA), di-octadecyl-amido-glycyl-spermine (DOGS), Dioleoylethyl-phosphocholine (DOEPC), diphytanoylphosphatidylethanolamine (DPhPE), dipalmitoylphosphatidylethanolamine (DPPE), 3,β-(1-ornithine amido carbamoyl) cholesterol (O-Chol), palmitoyloleoylphosphatidylethanolamine (POPE), or dipalmitoyloleoylphosphatidylglycerol (DPPG), or any mixture thereof.

In a further embodiment, the branched structure polypeptide composition or complex may contain one or more pharmaceutically acceptable carriers, buffers, diluents, excipients, or any combination thereof.

In a further embodiment, the carrier is in solid or liquid form, based on the administration method selected. The administration routes include, for example, intradermal or transdermal, oral, parenteral, including subcutaneous, intramuscular, or intravenous injections, topical or intranasal routes. The dosage of the invention is determined based on animal models. These models include humans and animals, including but not limited to, livestock such as feline or canine subjects, farm animals such as, but not limited to, cows, horses, goats, sheep, and pigs, research animals such as mice, rats, rabbits, goats, sheep, pigs, dogs, cats, etc., poultry such as chickens, turkeys, songbirds, and humans, goats, cows, pigs, dogs, cats, monkeys, apes, or rodents including mice, hamsters, and rabbits.

According to yet another aspect of the invention, the branched structure polypeptide composition or complex described in the technical solution is used in the preparation for the prevention, treatment, and/or improvement of diseases selected from the following: cancer or tumor diseases, viral or bacterial infections, genetic diseases, respiratory diseases, cardiovascular diseases, neurological diseases, digestive diseases, bone diseases, connective tissue diseases, immune deficiency diseases, endocrine diseases, eye diseases, and ear diseases.

According to yet another aspect of the invention, the branched structure polypeptide composition or complex described in the technical solution is used for the prevention, treatment, and/or improvement of animal diseases, used in veterinary pharmaceutical compositions, or as an animal vaccine.

The branched structure polypeptide or composition described in the technical solution of the present invention is capable of delivering RNA into animal/human cells, tissues, or individuals in a kit.

Preferably, the branched structure polypeptide described in the present invention can be selected from, but is not limited to, the following subset of formulas, including one or more combinations of them, with amino acid sequences as set forth in SEQ ID NO: 1-SEQ ID NO: 27 and as shown in Table 1, wherein a disulfide bond is formed between two cysteine (Cys) residues in the sequence:

TABLE 1

| Sequence | SEQ ID NOs: 1 | K(LSLLSLC)KKK(LSLLSLC) |
|---|---|---|
| 1 | and 13 | |
| Sequence | SEQ ID NOs: 2 | K(LLALLAC)KK(KS)KK(LLALLAC) |
| 2 | and 14 | |
| Sequence | SEQ ID NOs: 3 | K(LLAC)KK(KD)KK(KD)KK(LLAC) |
| 3 | and 15 | |
| Sequence | SEQ ID NO: 4 | K(LLA)KK(KRC)KK(LLA)KK(KRC)KK(LLA) |
| 4 | | |
| Sequence | SEQ ID NOs: 5 | K(LAALAALAALAALAAC)KKKK(LAALAALAALAALAAC) |
| 5 | and 16 | |
| Sequence | SEQ ID NOs: 6, | K(LSALSALSALSAC)KK(KKKKK)KK(LSALSALSALSAC) |
| 6 | 17, and 18 | |
| Sequence | SEQ ID NOs: 7, | K(LAALAALAAC)KK(KAKAK)KK(KAKAK)KK(LAALAALAAC) |
| 7 | 19, and 20 | |
| Sequence | SEQ ID NOs: 8, | K(LAALAALAA)KK(KRKRRC)KK(LAALAALAA)KK(KRKRRC)KK |
| 8 | 21, and 22 | (LAALAALAA) |
| Sequence | SEQ ID NOs: 9 | K(LAALAALAALAALAALAALAAC)KKKK(LAALAALAALAALAALAALAAC) |
| 9 | and 23 | |
| Sequence | SEQ ID NOs: | K(LAALAALAALAALAALAALAAC)KK(KSKKSKKSKKSKKSK) |
| 10 | 10, 23, and 24 | KK(LAALAALAALAALAALAALAAC) |
| Sequence | SEQ ID NOS: | K |
| 11 | 11, 16, and 25 | (LAALAALAALAALAAC)KK(KDKDKDKDKDKD)KK(KDKDKDKDKDKD)KK |
| | | (LAALAALAALAALAAC) |
| Sequence | SEQ ID NOS: | K(LAALAALAALAALAA)KK(KSKKSKKSKC)KK(LAALAALAALAALAA)KK |
| 12 | 12, 26, and 27 | (KSKKSKKSKC)KK(LAALAALAALAALAA) |

According to the present invention, first, the branched structure polypeptides are prepared through solid-phase synthesis. Then the polypeptides are mixed with mRNAs, followed by lipids, in a two-step process to form a nanoparticle complex. The physicochemical property characterization and in vitro and in vivo experimental validation are then performed. The main obstacle to peptide delivery in the present invention is the degradation of peptides. Cyclization can reduce protein cleavage, and branched peptides have the characteristic of improving half-life. The current strategy for solving the stability of peptide drugs is to conjugate them with carriers (such as liposomes and solid nanoparticles), or to construct multi-branched polypeptides to form nanodevices with specificity and stability. Branched polypeptides have a defined chemical structure and are more stable compared to linear peptides. The objective is to provide a structurally stable branched polypeptide carrier for efficient nucleic acid delivery.

The technical solution of the multi-branched polypeptide composition of the present invention provides a multi-branched polypeptide with high gene delivery efficiency, structural stability, and good biocompatibility, along with its variations. The technical solution of the present invention can efficiently deliver nucleic acids to tissues and organs in vivo to achieve targeted therapy. Compared to existing delivery carriers, the invention offers a series of beneficial technical effects. Polypeptides have several times the conformational flexibility and affinity of large molecular drugs (proteins and antibodies). Multi-branched polypeptides have long-term in vivo stability, while maintaining strong affinity and minimal toxicity.

The multi-branched polypeptide described in the present invention, as a new generation of biomaterials, possesses superior biological and chemical activity. Therefore, through the rational design of the amino acid sequence of the polypeptide, the polypeptides can form stable nano-complexes or nanoparticles with nucleic acid molecules through electrostatic interactions, enabling the delivery of nucleic acid drugs and their stable release within cells, enhancing the activity of nucleic acid drugs.

The preparation of the branched polypeptide composition in the present invention is relatively controllable. The branched polypeptide composition has good biodegradability and safety, low side effects, and enhanced therapeutic index. The composition exhibits good biological response effects and can effectively carry nucleic acid drugs into cells in tissues and organs in vivo, releasing the nucleic acid and generating therapeutic effects through endosomal escape.

The branched polypeptide composition in the present invention effectively enhances the structural robustness, cell membrane permeability, and metabolic stability of the polypeptide molecules by the regular arrangement of the amino acid sequence and the disulfide bond coupling technology. This provides an optimized technical solution for how to safely and efficiently deliver specific or normal nucleic acid sequences into the cells or tissues of patients or animals in molecular medicine therapies.

### BRIEF DESCRIPTION OF THE DRAWINGS

The following drawings further illustrate the present invention, and should not be construed as limiting the scope of the invention.

FIG. 1 shows the liquid chromatography graphs of two branched polypeptides.

FIG. 2 shows the mass spectrometry graphs of two branched polypeptides.

FIG. 3 shows the particle size distribution graphs of the branched polypeptide/lipid/mRNA complex formulations.

FIG. 4 shows the gel electrophoresis graphs of the branched polypeptide/lipid/mRNA complex formulation.

FIG. 5 shows the schematic diagrams of the branched polypeptide/lipid/mRNA complex formulations transfecting 293T cells and expressing the target fluorescent protein.

FIG. 6 shows the in vivo delivery of mRNA by the branched polypeptide/lipid/mRNA complex formulations in mice and the expression of the target gene protein.

FIG. 7 shows the gel electrophoresis graph of the branched-chain polypeptide and mRNA formulations.

FIG. 8 shows the fluorescent expression image of the branched polypeptide/lipid/mRNA complex formulations transfecting 293T cells.

FIG. 9 shows the toxicity graphs of the branched polypeptide/lipid/mRNA complex formulations in 293T cells.

FIG. 10 shows the expression of luciferase in vivo in mice after administration of the branched polypeptide/lipid/mRNA complex formulations.

### DETAILED DESCRIPTION

More detailed description of the present invention is provided through the illustrative examples and figures, to help those skilled in the art understand the present invention and help define the scope of protection of the present invention.

### EXAMPLE 1: PREPARATION OF THE DELIVERY CARRIER

### Step 1: Preparation of Peptide Resin

1) Select the starting resin for solid-phase synthesis based on the target sequence, and weigh the corresponding starting resin based on the resin substitution level and the scale of the synthesis. The formula for calculating the weight of the starting resin is as follows:

The weight of the starting resin (g)=Synthesis scale (mmol)/Resin substitution degree (mmol/g)

2) Place the weighed starting resin into a 100 mL reaction column. Add DCM (dichloromethane) or DMF (N,N-dimethylformamide) at a ratio of 8-10 mL per gram of resin, or a mixture of both solvents. Nitrogen gas is bubbled through for swelling for about 30 minutes. After that, the solvent is drained, and the resin is washed three times with DMF.

3) Add DBLK (20% piperidine/DMF mixture) solution to the reactor to remove the Fmoc protecting group. To ensure complete removal, this step is performed twice, for 5 minutes and 15 minutes, respectively.

4) After the Fmoc protection group is removed, a positive result is is obtained using ninhydrin color test (color development indicates positive result).

5) After the ninhydrin test, wash the resin with DMF 6 times, each washing for 1-2 minutes.

6) After cleaning, add the amino acids to be coupled in the target peptide sequence along with solid and liquid condensation reagents to carry out the coupling reaction. The reaction time is 1-2 hours (the condensation reagents used in this experiment are HBTU (benzotriazol-1-yl-oxytris(dimethylamino)phosphonium hexafluorophosphate)/DIEA (N,N-diisopropylethylamine), the reaction time is 1 hour).

7) After the reaction, a small amount of resin is taken for ninhydrin color test to obtain a negative result (negative means no color development). If a positive result is obtained, it means that some free amines are still present, indicating incomplete coupling. In this case, further reactants need to be added or repeatedly added until the reaction is complete.

8) After the reaction is complete, wash the resin three times with DMF and drain the washing solution. Repeat steps 3)-7) until the final amino acid of the target peptide sequence is coupled (solid-phase synthesis is generally carried out from the C-terminal to the N-terminal sequentially).

9) Once the entire peptide sequence has been coupled and the final Fmoc group removed, add methanol to shrink and dry the resin to obtain the peptide resin (this experiment involves two methanol shrinkage steps, each for approximately 10 minutes).

### Step 2: Preparation of Crude Peptide

1) Weigh the peptide resin from step 1, and add cleavage reagent to cleave the peptide from the resin. The purpose of cleavage is to remove both the carrier resin and the side-chain protecting groups of the amino acids in the sequence. The ratio and cleavage time of the cleavage reagent are selected according to the length of the peptide sequence and the complexity of the protecting groups used. (In this experiment, the cleavage reagent ratio is V_{TFA}:V_{phenylmethanethiol}:V_{phenylmethanol}:V_{EDT}=90:5:3:2, and the cleavage time is 2 hours).

2) After cleavage, filter out the resin and retain the filtrate. Add anhydrous ether to the filtrate at a ratio of 1 mL filtrate to 10 mL ether, and allow the mixture to settle for 20-30 minutes (20 minutes for this experiment).

3) After settling, centrifuge to remove the supernatant and obtain the filter cake. Add anhydrous ether again and repeat the washing and centrifuging process three times to thoroughly clean the filter cake.

4) After washing, place the filter cake in a drying chamber and vacuum dry to obtain crude peptide.

### Step 3: Peptide Cyclization and Final Product Preparation

1) Take the crude peptide from Step 2, grind it finely, and dissolve it in an appropriate solvent (water or a mixture of water and acetonitrile) to obtain a clear solution for analysis.

2) According to the requirements of the peptide, if cyclization is not needed, select appropriate gradient elution conditions for purification by preparative chromatography to further purify the sample. If cyclization is required, choose appropriate cyclization reagents for cyclization, followed by purification (hydrogen peroxide or iodine is used for cyclization in this experiment).

3) After one or more rounds of purification to achieve the desired purity, concentrate the sample by rotary evaporation and then freeze-dry under vacuum.

4) After lyophilization, the final peptide product is obtained. The sample is analyzed by HPLC (high-performance liquid chromatography) and identified by MS (mass spectrometry).

5) Once HPLC purity and molecular weight determination by mass spectrometry are confirmed, the product is stored (the storage condition of this experiment is −20° C.) for further experiments.

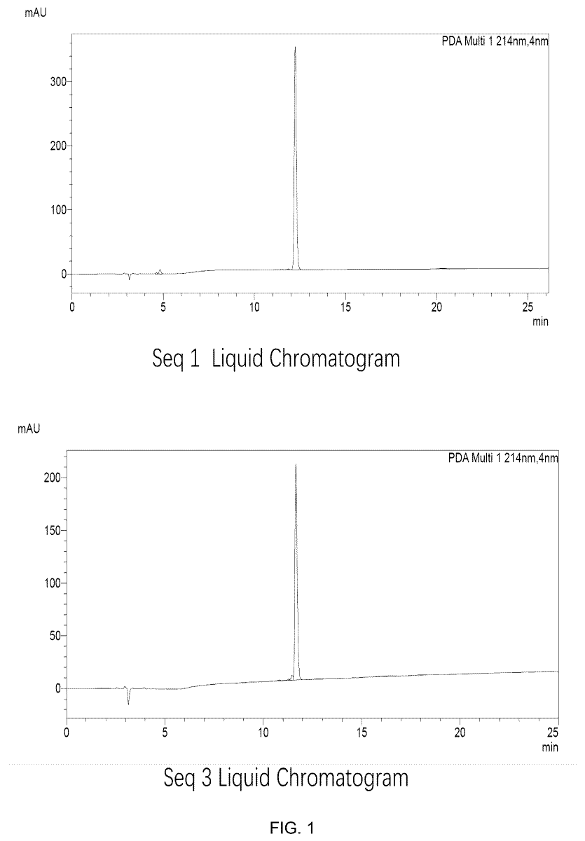

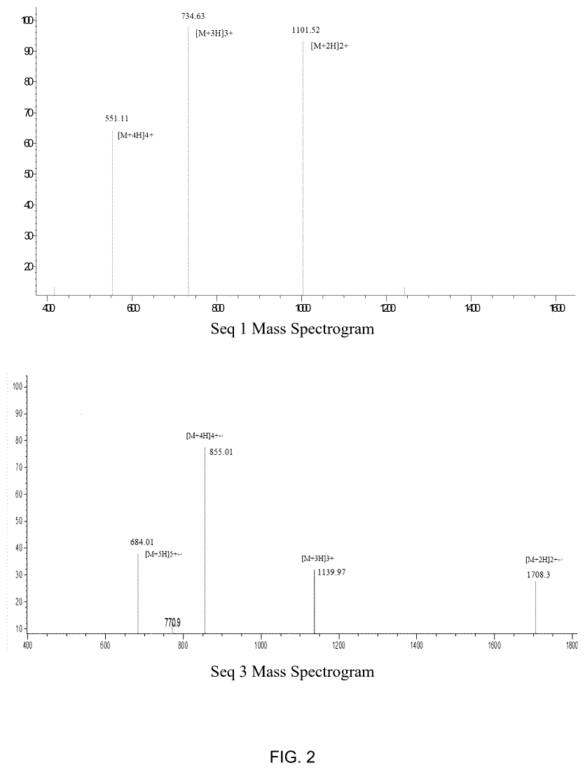

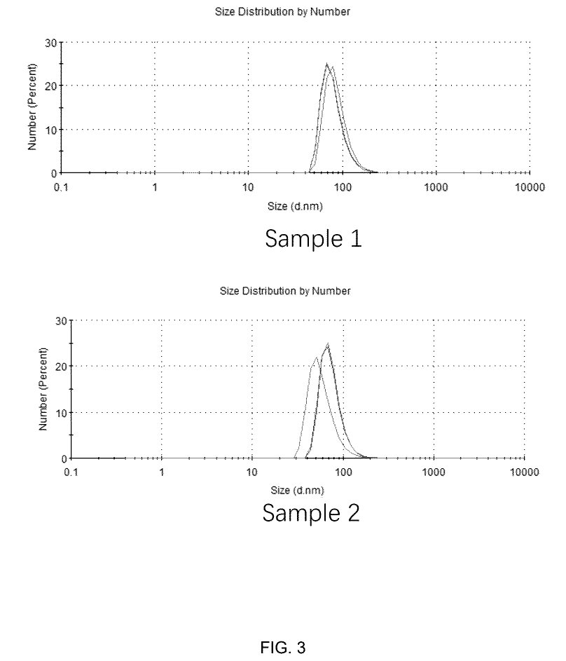

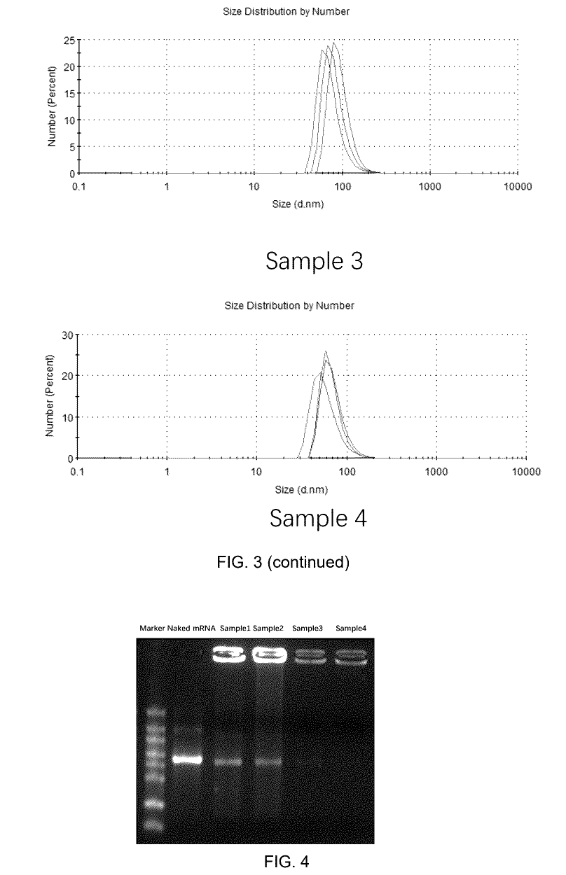

As shown in FIG. 1, the purity of Branched Polypeptide Sequences 1 and 3 are 97% and 95%, respectively. As indicated in FIG. 2, the molecular weight matches the theoretical molecular weight, confirming that the synthesized branched polypeptides meet the experimental requirements.

### EXAMPLE 2: PREPARATION METHOD OF NUCLEIC ACID DELIVERY CARRIER

First, four kinds of complex formulations of branched polypeptides (Sequences 1-4) with mRNA were prepared. The mass ratio of branched polypeptide to mRNA ranged from 30:1 to 1:30. Then, ionizable cationic lipids/neutral phospholipids/cholesterol/PEG-modified lipids in a molar ratio of 20-50:5-25:10-50:0.5-15 were mixed with the complex formulation solution from the first step at a mass ratio of 1:20 to 20:1. This mixture was further combined using a microfluidic chip to form complex particles. Ethanol was removed by ultrafiltration, and the final lipid/branched polypeptide/mRNA complex formulation was collected. In Sample 1, the mass ratio of Polypeptide Sequence 1 to mRNA is 1:1. Dlin-MC3-DMA, DSPC, cholesterol, and PEG-DMG were dissolved in ethanol and mixed with the complex formulation solution from the first step at a 1:8 mass ratio. In Sample 2, the mass ratio of Polypeptide Sequence 1 to mRNA is 1:1. Dlin-MC3-DMA, DSPC, cholesterol, and PEG-DMG were dissolved in ethanol and mixed with the complex formulation solution from the first step at a 1:10 mass ratio. In Sample 3, the mass ratio of Polypeptide Sequence 1 to mRNA is 1:1. Dlin-MC3-DMA, DSPC, cholesterol, and PEG-DMG were dissolved in ethanol and mixed with the complex formulation solution from the first step at a 1:12 mass ratio. In Sample 4, the mass ratio of Peptide Sequence 1 with mRNA is 1:1. Dlin-MC3-DMA, DSPC, cholesterol, and PEG-DMG were dissolved in ethanol and mixed with the complex formulation solution from the first step at a 1:15 mass ratio.

### EXAMPLE 3: PARTICLE SIZE MEASUREMENT EXPERIMENT

The Malvern Zeta Sizer Nano ZS (Malvern Nano Particle Size Analyzer) was used to measure the particle size (Size) and polydispersity index (PDI) of Samples 1-4 by dynamic light scattering (DLS). The results showed that the lipid/branched polypeptide/nucleic acid complex particles had a particle size of approximately 100 nm, with a PDI around 0.2, indicating that the formulation had well-controlled particle sizes.

TABLE 2

| | | Polydispersity Index |
|---|---|---|
| Experiment No. | Particle Size (nm) | (PDI) |
| 1 | 130.3 | 0.138 |
| 2 | 128.6 | 0.296 |
| 3 | 142.9 | 0.211 |
| 4 | 125.3 | 0.195 |

### EXAMPLE 4: AGAROSE GEL ELECTROPHORESIS EXPERIMENT

To investigate the binding ability between the branched polypeptide and the nucleic acid solution, agarose gel electrophoresis was performed.

1) Dissolve 1.5 g of agarose in 150 mL of 1×TBE buffer to prepare a 1% gel.

2) Add RNA fluorescence dye, mix thoroughly, and pour into the gel plate. The lipid/branched polypeptide/nucleic acid complex formulation was mixed with 2× buffer at a 1:1 ratio and set aside. Add an appropriate amount of TBE solution to the electrophoresis tank.

3) Set the voltage to 120V and run for 20 minutes.

The results showed that when the sample well fluoresced brightly, it indicated that mRNA was completely encapsulated. When no fluorescence was observed, or distinct RNA bands appeared outside the sample well, it indicated that mRNA was not encapsulated. As shown in FIG. 4, Samples 3 and 4 could completely encapsulate the nucleic acid.

### EXAMPLE 5: IN VITRO FLUORESCENT TRANSFECTION EXPERIMENT

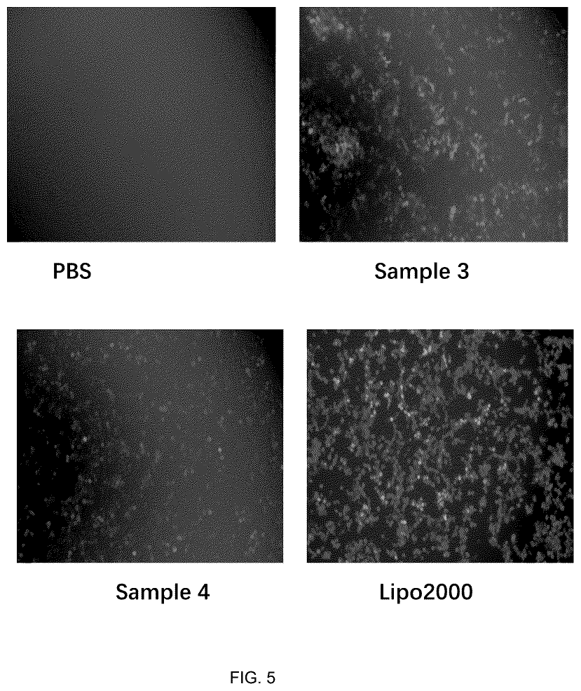

293T cells were seeded onto 24-well plates with 1 mL of culture medium per well and a cell density of 1×10\hat{ }5 cells/well. After 12 hours of culture until the cells adhered to the surface, 1 μg of mRNA formulation was added to 400 μL of serum-free medium and mixed. The positive control group was 3 μL of Lipofectamine 2000 and 1 μg of EGFP added to 400 μL of serum-free medium, mixed, and incubated for 10 minutes, followed by a supplement of 600 μL of DMEM complete medium. The negative control group was the naked mRNA group, in which 1 μg of mRNA was added to 1 mL of serum-free medium, imaged after 20 hours using a fluorescence microscope. As shown in FIG. 5, both the Lipofectamine 2000 group and the nucleic acid delivery carrier group exhibited green fluorescence signals, while the naked mRNA group showed no significant green fluorescence. This indicates that the lipid/branched polypeptide/nucleic acid complex formulation can effectively carry mRNA into cells for transfection and express the target fluorescent protein.

### EXAMPLE 6: IN VIVO EXPRESSION OF TARGET FLUORESCENT PROTEIN IN MICE

BALB/c female mice aged 6-8 weeks were used in the experiment. The lipid/branched polypeptide/nucleic acid complex formulation Sample 3 encapsulating mRNA encoding luciferase was injected intramuscularly at a dose of 100 μL (0.1 μg/μL) into each mouse. Fourteen hours after the injection, substrate Luciferin was injected intraperitoneally, and in vivo imaging of small animals was performed. After the substrate was exhausted, substrate Luciferin was re-injected, and the mice were dissected to observe the spleen, liver, and lung tissues.

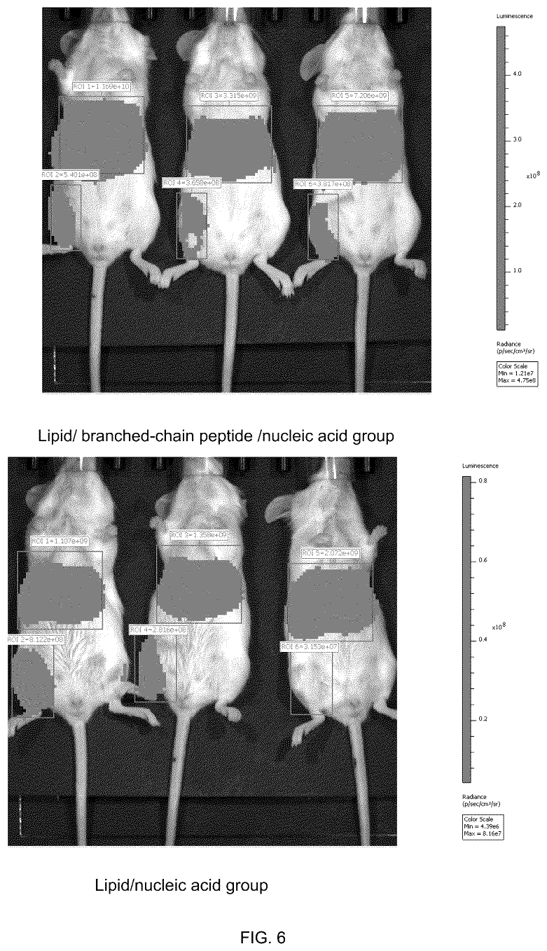

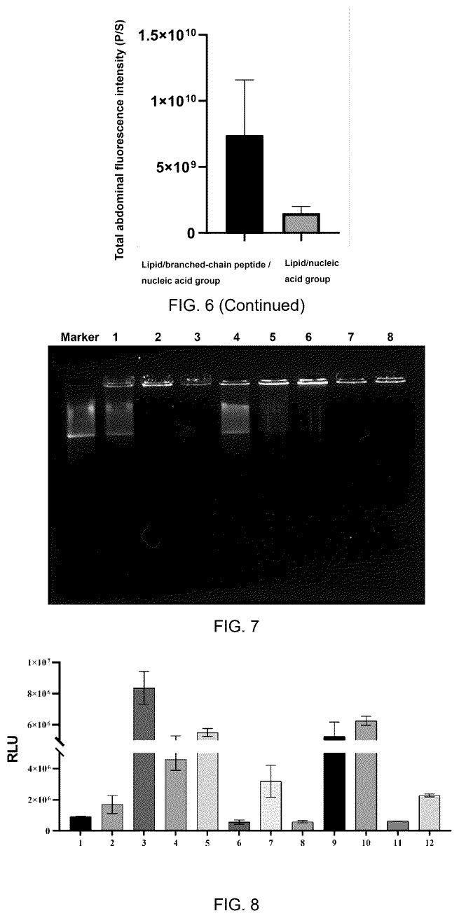

As shown in FIG. 6, mice in the lipid/branched polypeptide/nucleic acid complex formulation group exhibited significant fluorescence in their bodies. Post-mortem examination of the organs revealed that fluorescence was mainly concentrated in the spleen and liver tissue. This demonstrates that the lipid/branched polypeptide/nucleic acid complex formulation effectively delivers mRNA and expresses the target gene protein in vivo.

### EXAMPLE 7: AGAROSE GEL ELECTROPHORESIS EXPERIMENT (FURTHER ANALYSIS)

A 1.5% agarose gel was prepared by dissolving agarose in a TBE buffer using a microwave. After adding the sub-green dye, the solution was poured into the gel plate, and a comb was inserted. Polypeptide Sequences 9-12 samples were each mixed with mRNA and then mixed with the sample loading buffer in a 1:1 ratio and set aside after thorough mixing. The gel was loaded with Marker and the samples, then subjected to electrophoresis at 190 V for 20 minutes, and images were captured using a gel imaging system. As shown in FIG. 7, the bands from left to right represent Marker, Polypeptide Sequence 9 peptide with mRNA at a 1:1 mass ratio, Polypeptide Sequence 9 peptide with mRNA at a 1:2 mass ratio, Polypeptide Sequence 10 with mRNA at a 1:1 mass ratio, Polypeptide Sequence 10 with mRNA at a 1:2 mass ratio), Polypeptide Sequence 11 with mRNA at a 1:1 mass ratio, Polypeptide Sequence 11 with mRNA at a 1:2 mass ratio), Polypeptide Sequence 12 with mRNA at a 1:1 mass ratio, and Polypeptide Sequence 12 with mRNA at a 1:2 mass ratio. The results indicate that all the complexes remained within the sample wells, demonstrating that the peptide carriers encapsulated the mRNA, effectively protecting it from degradation.

### EXAMPLE 8: PREPARATION OF NUCLEIC ACID DELIVERY CARRIER

Polypeptides Sequences 5-12 were each mixed with mRNA at a 1:1 mass ratio. Dlin-MC3-DMA, DSPC, cholesterol, and PEG-DMG were dissolved in ethanol and mixed with each of the complex formulations from the first step at a 1:12 mass ratio to obtain the nanoformulations. These formulations were named Samples 5-12, respectively.

### EXAMPLE 9: PARTICLE SIZE MEASUREMENT

Using the Malvern Zetasizer NanoZS, the particle size, polydispersity index (PDI), and zeta potential (Zeta P) of the Samples 5-12 nanoformulations were measured by dynamic light scattering. The results are shown in Table 3, indicating that the particle sizes of the formulations are around 100 nm, with zeta potentials mostly around 20 mV. The PDI values range from 0.1 to 0.4.

TABLE 3

| | Particle Size | Polydispersity Index | Zeta Potential |
|---|---|---|---|
| Sample Number | (nm) | (PDI) | (mV) |
| 5 | 114.11 | 0.31 | 21.6 |
| 6 | 132.43 | 0.33 | 23.53 |
| 7 | 110.90 | 0.17 | 21.73 |
| 8 | 154.17 | 0.22 | −0.06 |
| 9 | 114.23 | 0.26 | 22.57 |
| 10 | 151.23 | 0.41 | 23.10 |
| 11 | 109.20 | 0.27 | 20.03 |
| 12 | 184.23 | 0.22 | 21.67 |

### EXAMPLE 10: IN VITRO CELL TRANSFECTION EXPERIMENT

293T cells were seeded in 96-well plates at a density of 3×10\hat{ }4 cells per well and cultured for 24 hours until they adhered to surface. Nanoformulations Samples 1-12 were each added to the cells. After 24 hours of incubation, the fluorescent protein expression of the cells were detected using the Bright-Lite™ Luciferase Assay System. The luminescence of each well was measured with a microplate reader. FIG. 8 shows that the nanoformulations can successfully carry mRNA into 293T cells for transfection, resulting in fluorescent protein expression, indicating that the branched polypeptides can effectively carry nucleic acids into cells. The branched polypeptide lipid nanoparticle complexes demonstrated good expression levels at the cellular level.

### EXAMPLE 11: CELL CYTOTOXICITY EXPERIMENT

HEK-293T cells were seeded in 96-well plates at a density of 3,000 cells per well and cultured overnight at 37° C. and 5% CO₂. After 24 hours, the media were discarded, and media with different concentrations of drugs were added. Commercially available MC3-LNP was used as the control. Nanoformulations Samples 1-3 prepared as described in Example 2 were added to the cells. After another 24 hours of incubation, 10 μL of CCK-8 solution was added to each well, and after 2 hours of incubation at 37° C., the absorbance at 450 nm was measured using a microplate reader to calculate cell viability. The cytotoxicity results are shown in FIG. 9. Under these experimental conditions, the branched polypeptide lipid nanoparticle complexes demonstrated lower cytotoxicity compared to the commercial MC3-LNP, showing better biocompatibility and significantly higher cell proliferation. The OD values for Samples 1, 2, and 3 were 0.71, 1.01, and 0.90, respectively, while the OD value for MC3-LNP was 0.65.

### EXAMPLE 12: IN VIVO FLUORESCENT PROTEIN EXPRESSION EXPERIMENT IN MICE

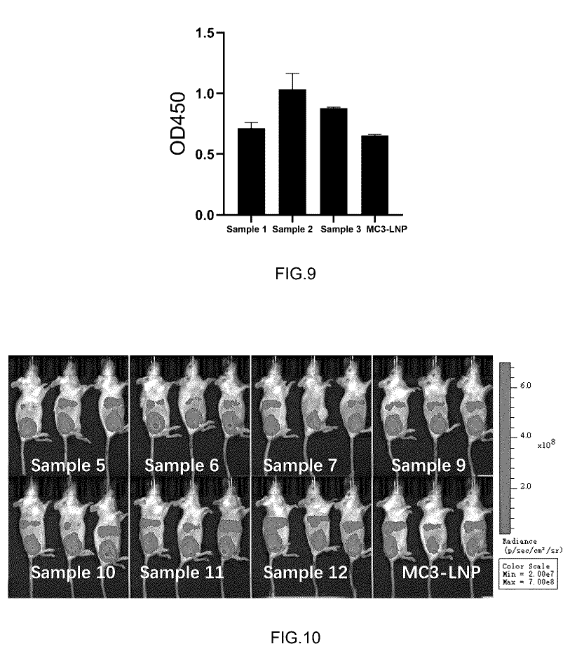

6-8-week-old female SPF-grade BALB/c mice (n=3) were used in this experiment, housed in SPF-level animal facilities with a 12/12-hour light/dark cycle, a temperature range of 20-26° C., and humidity between 40-70%, with plenty of food and water. Each of the mice was administered a 100 μL (0.05 μg/μL) dose intramuscularly, with formulations prepared according to Example 8. Six hours after injection, 200 μL of 15 mg/mL D-luciferin potassium salt was administered intraperitoneally, and the mice were placed in a supine position on the imaging platform. After 5 minutes, in vivo imaging was performed. After imaging, D-luciferin potassium salt was re-injected, and after 5 minutes, the mice were euthanized, and their hearts, livers, spleens, lungs, and kidneys were collected for organ imaging. As shown in FIG. 10, compared to the commercial MC3-LNP lipid formulation, the mice in Sample groups 5, 6, 7, 9, 10, 11, and 12 exhibited significant fluorescence expression in vivo, indicating that the branched polypeptide lipid nanoparticle complexes had better expression levels in vivo.

What is claimed is:

1. A branched structure polypeptide having one of the following general sequence formulas: 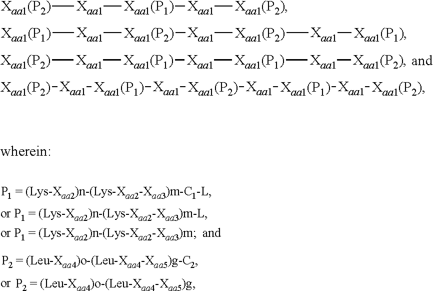
wherein:
Xₐₐ₁ is Aspartic acid (Asp), Glutamic acid (Glu), or Lysine (Lys),
Xₐₐ₂, Xₐₐ₃ each is any of Aspartic acid (Asp), Glutamic acid (Glu), Lysine (Lys), Arginine (Arg), Alanine (Ala), Glycine (Gly), Serine (Ser), Asparagine (Asn), and Glutamine (Gln);
Xₐₐ₄, Xₐₐ₅ each is any of Glycine (Gly), Serine (Ser), Tyrosine (Tyr), Threonine (Thr), Alanine (Ala), Valine (Val), Leucine (Leu), Isoleucine (Ile), Proline (Pro), Phenylalanine (Phe), Tryptophan (Trp), and Methionine (Met);
C₁ and C₂ are Cysteine (Cys);
L is optional and, where present, can be any of spermine, spermidine, polylysine, polyarginine, MPG peptide, HIV-binding peptide Tat, SAP peptide, MAP peptide, KALA peptide, FGF peptide, HSV peptide, RGD peptide, GLP-1 peptide, PEP-1 peptide, signal peptides or signal sequences, cytokines, growth factors, hormones, small molecular drugs, carbohydrates, therapeutic active proteins or peptides, antigens or antigen epitopes, antibodies, antibody receptor ligands, synthetic ligands, receptor inhibitors or antagonists, immunostimulatory proteins or peptides, protamine, nucleolar proteins, transferrin, polycationic peptide sequences, polyamines, peptide nucleic acids, nucleic acid embedding agents, nuclear localization signals or sequences (NLS), cell-penetrating peptides, mannose, galactose, small molecular agonists, poly-L-lysine (PLL), basic peptides, calcitonin peptides, lactoferrin, histones, and VP22-derived or similar peptides, or VP22 (herpes simplex); and
m, n, o, g may independently be selected from any of the values 0, 1, 2, 3, 4, 5, 6, 7, 8, 9, or 10, and
wherein:
the branched structure polypeptide comprises a length of 20-150 amino acid residues;
the branched structure comprises a two-branch, three-branch, four-branch, or five-branch configuration; and
the branched structure polypeptide comprises at least one disulfide bridge.

2. A branched structure polypeptide composition comprising one or more of the branched structure polypeptides of claim 1.

3. A branched structure polypeptide-nucleic acid complex, wherein the complex is obtained by mixing the branched-chain polypeptide composition of claim 2 with one or more nucleic acids.

4. The branched structure polypeptide-nucleic acid complex of claim 3, wherein the nucleic acid comprises naturally-occurring DNA, RNA, or artificially designed and synthesized DNA or RNA, such as small interfering RNA (siRNA), messenger RNA (mRNA), microRNA, small activating RNA (saRNA), antisense RNA, or immunostimulatory nucleic acids.

5. The branched structure polypeptide-nucleic acid complex of claim 4, wherein the nucleic acid comprises messenger RNA (mRNA).

6. The branched structure polypeptide-nucleic acid complex of claim 4, wherein the branched structure polypeptide-nucleic acid complex can efficiently carry nucleic acids across cell membranes in animals and humans and release them.

7. The branched structure polypeptide-nucleic acid complex of claim 6, wherein the nucleic acid comprises messenger RNA (mRNA).

8. The branched structure polypeptide-nucleic acid complex of claim 3, wherein the branched structure polypeptide-nucleic acid complex is used in combination with drugs or therapeutic agents selected from peptides, antibodies, enzymes, hormones, antitumor drugs, antilipidemic drugs, antibiotics, anti-inflammatory agents, cytotoxic agents, cell growth inhibitors, immunomodulators, and neuroactive agents.

9. The branched structure polypeptide-nucleic acid complex of claim 3, wherein the branched structure polypeptide-nucleic acid complex comprises a complex formed with a branched structure polypeptide and a C12-C18 alkyl group or a C6-C18 alkyl containing an olefinic bond.

10. The branched structure polypeptide composition of claim 2, wherein the branched-chain polypeptide composition comprises a surfactant, liposome, lipidoid, ethosome, transporter, phospholipid, or any combination thereof.

11. The branched structure polypeptide-nucleic acid complex of claim 3, wherein the branched polypeptide-nucleic acid complex comprises a surfactant, liposome, lipidoid, ethosome, transporter, phospholipid, or any combination thereof.

12. The branched structure polypeptide composition of claim 2, wherein the branched polypeptide-nucleic acid complex comprises one or more combinations with pharmaceutically acceptable carriers, buffers, diluents, and excipients.

13. The branched structure polypeptide-nucleic acid complex of claim 3, wherein the branched polypeptide-nucleic acid complex comprises one or more combinations with pharmaceutically acceptable carriers, buffers, diluents, and excipients.

14. A use of the branched structure polypeptide composition of claim 2 for preparing products to prevent, treat, or ameliorate diseases selected from cancers and tumors, viral and bacterial infections, genetic diseases, respiratory diseases, cardiovascular diseases, neurological disorders, digestive diseases, skeletal diseases, connective tissue disorders, immune deficiencies, endocrine diseases, ocular diseases, and auditory diseases.

15. A use of the branched structure polypeptide-nucleic acid complex of claim 3 for preparing products to prevent, treat, or ameliorate diseases selected from cancers and tumors, viral and bacterial infections, genetic diseases, respiratory diseases, cardiovascular diseases, neurological disorders, digestive diseases, skeletal diseases, connective tissue disorders, immune deficiencies, endocrine diseases, ocular diseases, and auditory diseases.

16. A use of the branched structure polypeptide composition of claim 2 for preparing products to prevent, treat, or ameliorate animal diseases, wherein the use is in veterinary drug formulations or in animal vaccines.

17. A use of the branched structure polypeptide-nucleic acid complex of claim 3 for preparing products to prevent, treat, or ameliorate animal diseases, wherein the use is in veterinary drug formulations or in animal vaccines.


SEQUENCE LISTING (27 sequences)

SEQ ID NO: 1 - protein, 4 aa, synthetic construct
  SITE 1: note = The side chain of the lysine is linked via an amide linkage to the C-terminus of SEQ ID NO: 13.
  SITE 4: note = The side chain of the lysine is linked via an amide linkage to the C-terminus of SEQ ID No: 13.
KKKK

SEQ ID NO: 2 - protein, 5 aa, synthetic construct
  SITE 1: note = The side chain of the lysine is linked via an amide linkage to the C-terminus of SEQ ID NO: 14.
  SITE 3: note = The side chain of the lysine is linked via an amide linkage to the C-terminus of the amino acid sequence SK.
  SITE 5: note = The side chain of the lysine is linked via an amide linkage to the C-terminus of SEQ ID NO:14.
KKKKK

SEQ ID NO: 3 - protein, 7 aa, synthetic construct
  SITE 1: note = The side chain of the lysine is linked via an amide linkage to the C-terminus of SEQ ID NO:15.
  SITE 3: note = The side chain of the lysine is linked via an amide linkage to the C-terminus of the amino acid sequence DK.
  SITE 5: note = The side chain of the lysine is linked via an amide linkage to the C-terminus of the amino acid sequence DK.
  SITE 7: note = The side chain of the lysine is linked via an amide linkage to the C-terminus of SEQ ID NO:15.
KKKKKKK

SEQ ID NO: 4 - protein, 9 aa, synthetic construct
  SITE 1: note = The side chain of the lysine is linked via an amide linkage to the C-terminus of the amino acid sequence ALL.
  SITE 5: note = The side chain of the lysine is linked via an amide linkage to the C-terminus of the amino acid sequence ALL.
  SITE 9: note = The side chain of the lysine is linked via an amide linkage to the C-terminus of the amino acid sequence ALL.
  SITE 3: note = The side chain of the lysine is linked via an amide linkage to the C-terminus of the amino acid sequence CRK.
  SITE 7: note = The side chain of the lysine is linked via an amide linkage to the C-terminus of the amino acid sequence CRK.
KKKKKKKKK

SEQ ID NO: 5 - protein, 5 aa, synthetic construct
  SITE 1: note = The side chain of the lysine is linked via an amide linkage to the C-terminus of SEQ ID NO: 16.
  SITE 5: note = The side chain of the lysine is linked via an amide linkage to the C-terminus of SEQ ID NO: 16.
KKKKK

SEQ ID NO: 6 - protein, 5 aa, synthetic construct
  SITE 1: note = The side chain of the lysine is linked via an amide linkage to the C-terminus of SEQ ID NO: 17.
  SITE 5: note = The side chain of the lysine is linked via an amide linkage to the C-terminus of SEQ ID NO: 17.
  SITE 3: note = The side chain of the lysine is linked via an amide linkage to the C-terminus of SEQ ID NO: 18.
KKKKK

SEQ ID NO: 7 - protein, 7 aa, synthetic construct
  SITE 1: note = The side chain of the lysine is linked via an amide linkage to the C-terminus of SEQ ID NO: 19.
  SITE 7: note = The side chain of the lysine is linked via an amide linkage to the C-terminus of SEQ ID NO: 19.
  SITE 3: note = The side chain of the lysine is linked via an amide linkage to the C-terminus of SEQ ID NO: 20.
  SITE 5: note = The side chain of the lysine is linked via an amide linkage to the C-terminus of SEQ ID NO: 20.
KKKKKKK

SEQ ID NO: 8 - protein, 9 aa, synthetic construct
  SITE 1: note = The side chain of the lysine is linked via an amide linkage to the C-terminus of SEQ ID NO: 21.
  SITE 5: note = The side chain of the lysine is linked via an amide linkage to the C-terminus of SEQ ID NO: 21.
  SITE 9: note = The side chain of the lysine is linked via an amide linkage to the C-terminus of SEQ ID NO: 21.
  SITE 3: note = The side chain of the lysine is linked via an amide linkage to the C-terminus of SEQ ID NO: 22.
  SITE 7: note = The side chain of the lysine is linked via an amide linkage to the C-terminus of SEQ ID NO: 22.
KKKKKKKKK

SEQ ID NO: 9 - protein, 5 aa, synthetic construct
  SITE 1: note = The side chain of the lysine is linked via an amide linkage to the C-terminus of SEQ ID NO: 23.
  SITE 5: note = The side chain of the lysine is linked via an amide linkage to the C-terminus of SEQ ID NO: 23.
KKKKK

SEQ ID NO: 10 - protein, 5 aa, synthetic construct
  SITE 1: note = The side chain of the lysine is linked via an amide linkage to the C-terminus of SEQ ID NO: 24.
  SITE 5: note = The side chain of the lysine is linked via an amide linkage to the C-terminus of SEQ ID NO: 24.
  SITE 3: note = The side chain of the lysine is linked via an amide linkage to the C-terminus of SEQ ID NO: 25.
KKKKK

SEQ ID NO: 11 - protein, 7 aa, synthetic construct
  SITE 1: note = The side chain of the lysine is linked via an amide linkage to the C-terminus of SEQ ID NO: 26.
  SITE 7: note = The side chain of the lysine is linked via an amide linkage to the C-terminus of SEQ ID NO: 26.
  SITE 3: note = The side chain of the lysine is linked via an amide linkage to the C-terminus of SEQ ID NO: 27.
  SITE 5: note = The side chain of the lysine is linked via an amide linkage to the C-terminus of SEQ ID NO: 27.
KKKKKKK

SEQ ID NO: 12 - protein, 9 aa, synthetic construct
  SITE 1: note = The side chain of the lysine is linked via an amide linkage to the C-terminus of SEQ ID NO: 28.
  SITE 9: note = The side chain of the lysine is linked via an amide linkage to the C-terminus of SEQ ID NO: 28.
  SITE 3: note = The side chain of the lysine is linked via an amide linkage to the C-terminus of SEQ ID NO: 29.
  SITE 7: note = The side chain of the lysine is linked via an amide linkage to the C-terminus of SEQ ID NO: 29.
  SITE 5: note = The side chain of the lysine is linked via an amide linkage to the C-terminus of SEQ ID NO: 30.
KKKKKKKKK

SEQ ID NO: 13 - protein, 7 aa, synthetic construct
  SITE 7: note = The leucine is linked via an amide linkage to the side chain of the lysine in positions #1 and #4 in SEQ ID NO: 1.
CLSLLSL

SEQ ID NO: 14 - protein, 7 aa, synthetic construct
  SITE 7: note = The leucine is linked via an amide linkage to the side chain of the lysine in positions #1 and #5 of SEQ ID NO: 2.
CALLALL

SEQ ID NO: 15 - protein, 4 aa, synthetic construct
  SITE 4: note = The leucine is linked via an amide linkage to the side chain of the lysine in positions #1 and #7 of SEQ ID NO: 3.
CALL

SEQ ID NO: 16 - protein, 16 aa, synthetic construct
  SITE 16: note = The leucine is linked via an amide linkage to the side chain of the lysine in positions #1 and #5 of SEQ ID NO: 5 and in positions #1 and #7 of SEQ ID NO: 11.
CAALAALAALAALAAL

SEQ ID NO: 17 - protein, 13 aa, synthetic construct
  SITE 13: note = The leucine is linked via an amide linkage to the side chain of the lysine in positions #1 and #5 of SEQ ID NO: 6.
CASLASLASLASL

SEQ ID NO: 18 - protein, 5 aa, unidentified
  SITE 3: note = The lysine is linked via an amide linkage to the side chain of the lysine in position #3 of SEQ ID NO: 6.
KKKKK

SEQ ID NO: 19 - protein, 10 aa, synthetic construct
  SITE 10: note = The leucine is linked via an amide linkage to the side chain of the lysine in positions #1 and #7 of SEQ ID NO: 7.
CAALAALAAL

SEQ ID NO: 20 - protein, 5 aa, synthetic construct
  SITE 5: note = The Lysine is linked via an amide linkage to the side chain of the lysine in positions #3 and #5 of SEQ ID NO: 7.
KAKAK

SEQ ID NO: 21 - protein, 9 aa, synthetic construct
  SITE 9: note = The leucine is linked via an amide linkage to the side chain of the lysine in positions #1, #5, and #9 of SEQ ID NO: 8.
AALAALAAL

SEQ ID NO: 22 - protein, 6 aa, synthetic construct
  SITE 6: note = The lysine is linked via an amide linkage to the side chain of the lysine in positions #3 and #7 of SEQ ID NO: 8.
CRRKRK

SEQ ID NO: 23 - protein, 22 aa, synthetic construct
  SITE 22: note = The leucine is linked via an amide linkage to the side chain of the lysine in positions #1 and #5 of SEQ ID NO: 9 and in positions #1 and #5 of SEQ ID NO: 10.
CAALAALAALAALAALAALAAL

SEQ ID NO: 24 - protein, 15 aa, synthetic construct
  SITE 15: note = The lysine is linked via an amide linkage to the side chain of the lysine in position #3 of SEQ ID NO: 10.
KSKKSKKSKKSKKSK

SEQ ID NO: 25 - protein, 12 aa, synthetic construct
  SITE 12: note = The lysine is linked via an amide linkage to the side chain of the lysine in positions #3and #5 of SEQ ID NO: 11.
DKDKDKDKDKDK

SEQ ID NO: 26 - protein, 15 aa, synthetic construct
  SITE 15: note = The leucine is linked via an amide linkage to the side chain of the lysine in positions #1, #5, and #9 of SEQ ID NO: 12.
AALAALAALAALAAL

SEQ ID NO: 27 - protein, 10 aa, synthetic construct
  SITE 10: note = The lysine is linked via an amide linkage to the side chain of the lysine in positions #3 and #7 of SEQ ID NO: 12.
CKSKKSKKSK